(19) United States
(12) Patent Application Publication
(10) Pub. No.: US 2021/0260364 A1
(43) Pub. Date: Aug. 26, 2021

(54) HIGH DENSITY NEURAL IMPLANTS FOR BRAIN-MACHINE INTERFACES

(71) Applicants: Brown University, Providence, RI (US); UNIVERSITY OF CALIFORNIA, SAN DIEGO, La Jolla, CA (US)

(72) Inventors: Peter Michael Asbeck, Del Mar, CA (US); Farah Laiwalla, Barrington, RI (US); Vincent Wingching Leung, San Diego, CA (US); Jihun Lee, Providence, RI (US); Lawrence Ernest Larson, Providence, RI (US); Patrick Mercier, San Diego, CA (US); Arto Nurmikko, Providence, RI (US); Ramesh Rao, La Jolla, CA (US)

(21) Appl. No.: 17/260,903

(22) PCT Filed: Jul. 16, 2019
(86) PCT No.: PCT/US2019/042051

§ 371 Date: Jan. 15, 2021

Related U.S. Application Data
(60) Provisional application No. 62/699,472, filed on Jul. 17, 2018.

(51) Int. Cl. A61N 1/05; A61N 1/36; A61N 1/372; A61N 1/378

(52) U.S. Cl. CPC A61N 1/0529; A61N 1/3787; A61N 1/37229; A61N 1/36125

(57) ABSTRACT

A system includes an implanted autonomous microchiplet communicatively linked to an epidermal skinpatch having a transceiver and a demodulator, an intermediate device communicatively linked to the epidermal skinpatch, the intermediate device configured to receive neural signals for decoding from the epidermal skinpatch and to send encoded signals for patterned stimulation to the epidermal skinpatch, and one or more external devices communicatively linked to intermediate device.

### CROSS REFERENCE TO RELATED APPLICATIONS

This application claims benefit from U.S. Provisional Patent Application Ser. No. 62/699,472, filed Jul. 17, 2018, which is incorporated by reference in its entirety.

### STATEMENT REGARDING GOVERNMENT INTEREST

This invention was made with government support under DARPA-16-09-NESD-FP-001. The government has certain rights in the invention.

### BACKGROUND OF THE INVENTION

The present invention relates generally to networks, and more particularly to high density neural implants for brain-machine interfaces.

In general, brain implants, often referred to as neural implants, are technological devices that connect directly to a biological subject's brain—usually placed on the surface of the brain, or inserted to the brain's cortex. A common purpose of modern brain implants is establishing a biomedical prosthesis circumventing areas in the brain that have become dysfunctional after a stroke or other head injuries. This includes sensory substitution, e.g., in vision. Other brain implants are used in animal experiments to record brain activity for advancement of neuroscientific understanding of the brain. These brain implants are designed to serve as an interface between native neural systems and external computing systems for the purpose of creating function Brain-computer interface “Neuroprosthesis” systems.

Current state-of-the-art Brain-Machine Interfaces (BMIs) rely on invasive “passive” microelectrode technologies, which are prohibitively challenging to scale beyond several hundred channels due to physical constraints.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a system including a distributed sensor system of complementary metal-oxide-semiconductor (CMOS) untethered microelectronic chiplets forming a network of individual neural interfacing nodes in a cortex for active neural recording and electrical microstimulation.

In another aspect, the invention features a system including a distributed sensor system of CMOS neurograins that provide a high density network of autonomous implantable neural sensors.

In still another aspect, the invention features system including an implanted autonomous microchiplet communicatively linked to an epidermal skinpatch having a transceiver and a demodulator, an intermediate device communicatively linked to the epidermal skinpatch, the intermediate device configured to receive neural signals for decoding from the epidermal skinpatch and to send encoded signals for patterned stimulation to the epidermal skinpatch, and one or more external devices communicatively linked to intermediate device.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 illustrates a structure of an exemplary deep neural network.

FIG. 2 illustrates an exemplary flow diagram.

FIG. 2A illustrates an exemplary training procedure for DNNS with reduced-precision parameters.

FIG. 2B illustrates exemplary ensemble processing.

FIG. 3 illustrates an exemplary hardware accelerator design (a) a single neuron and (b) the organization of neurons and hardware blocks.

FIG. 4 illustrates the classification error rate of the baseline floating-point network.

FIG. 5 is a block diagram of another exemplary hardware accelerator.

FIG. 6 shows an exemplary breakdown of power and area for the hardware accelerator.

FIG. 7 illustrates an exemplary plot.

FIGS. 8(a)-8(d) illustrate exemplary graphs.

FIGS. 9(a)-9(d) illustrate exemplary plots.

FIG. 10 illustrates an overview of a spacially-distributed cluster of adaptive, implanted neurograins and external telecommunications hub leading to a wearable neurocomputational unit.

FIGS. 11(a)-11(d) illustrate exemplary block diagrams.

FIG. 12 illustrates a top level block diagram 1200 of a wireless stimulating neurograin.

FIG. 13 illustrates an overview block diagram 1300 for an implementation of analog Gaussian-noise variance based voltage thresholding spike detection circuit.

FIG. 14 illustrates an exemplary wireless power transfer for a neurograin system.

FIG. 15 illustrates a time-division multiple access RF communication scheme.

FIG. 16 illustrates an overview of a fSM implemented on a neurograin ASIC.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

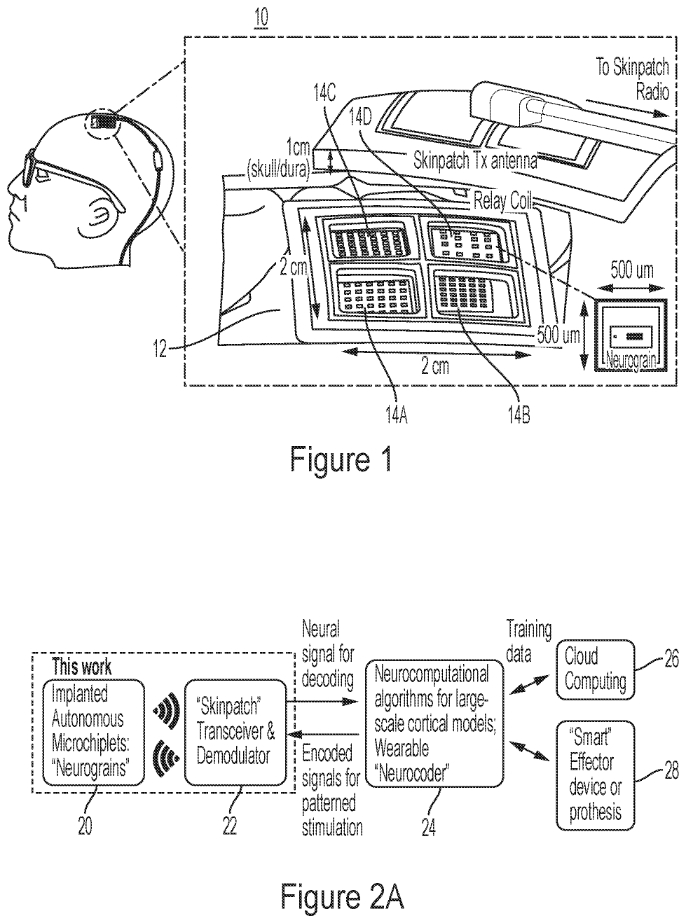

In FIG. 1, an exemplary cortical surface neurograin sensor system 10 includes a two-dimensional (2D) grid 12 of individual neurograin (0.5 mm×0.5 mm shown in inset) chips 14A, 14B, 14C, 14D embedded in a substrate. Coupling between a chip micro antenna and a transmit skinpatch antenna is optimized by a relay antenna. At a core of each neurograin chip is a 500 μm×500 μm ASIC. This chip contains a dedicated RF micro-antenna and all the associated analog front-end, networking, and backscatter circuits. The neurograin chips 14A, 14B, 14C, 14D are powered by inductive coupling at ˜1 GHz RF to the epidermal skinpatch antenna.

Each neurograin (“NG”) chip 14A, 14B, 14C, 14D has a unique ID, which is used to identify the chip. More specifically, a “Physically Uncloneable Function” (PUF) circuit that uses process variations in microelectronic fabrication to generate unique IDs for each NG chip 14A, 14B, 14C, 14D is used. These IDs are random numbers, and each NG chip 14A, 14B, 14C, 14D has a 60-bit PUF ID created during batch fabrication. A network discovery protocol enables the IDs to be communicated to a telecommunication hub, where the latter can then assign a slot to each NG chip 14A, 14B, 14C, 14D based on its ID in the telecommunication line up (according to the time-division multiple access protocol).

For the PUF address discovery, for example, a random search paradigm is used that expedites a discovery of one thousand neurograin chips (with 60-bit address each) to only four thousand process steps.

In one example, Amplitude-Shift-Keying-Pulse-Width-Modulation (ASK-PWM) for downlink communication is used. This technique to minimizes the communication overhead by using synchronized downlink across the network in a simultaneous instruction without the need to incorporate individual 60-bit addresses for each NG chip 14A, 14B, 14C, 14D into the downlink packet.

The system 10 uses a 3-coil electromagnetic coupling system. The introduction of a relay antenna between the skinpatch and the NG chips 14A, 14B, 14C, 14D concentrates flux and enhances electromagnetic coupling, significantly mitigating RF signal attenuation while enabling wide area coverage to power hundreds of NG chips within the coil perimeter. The wireless transfer efficiency from skinpatch to NG chip is increased from −48.7 dB to −30.3 dB according to HFSS simulations (in tissue medium). In one implementation, the antenna uses window-shaped transmit-relay antennas which measure two cm per edge, and are able to connect to up to 1000 neurograin chips simultaneously.

Backscattered transmitted data from the neurograin chips is BPSK modulated, and the skinpatch Tx-Rx antenna connects to the RF BPSK demodulator. The recovered demodulated data is then channeled via neurocomputational processing units to the functional prosthetic devices.

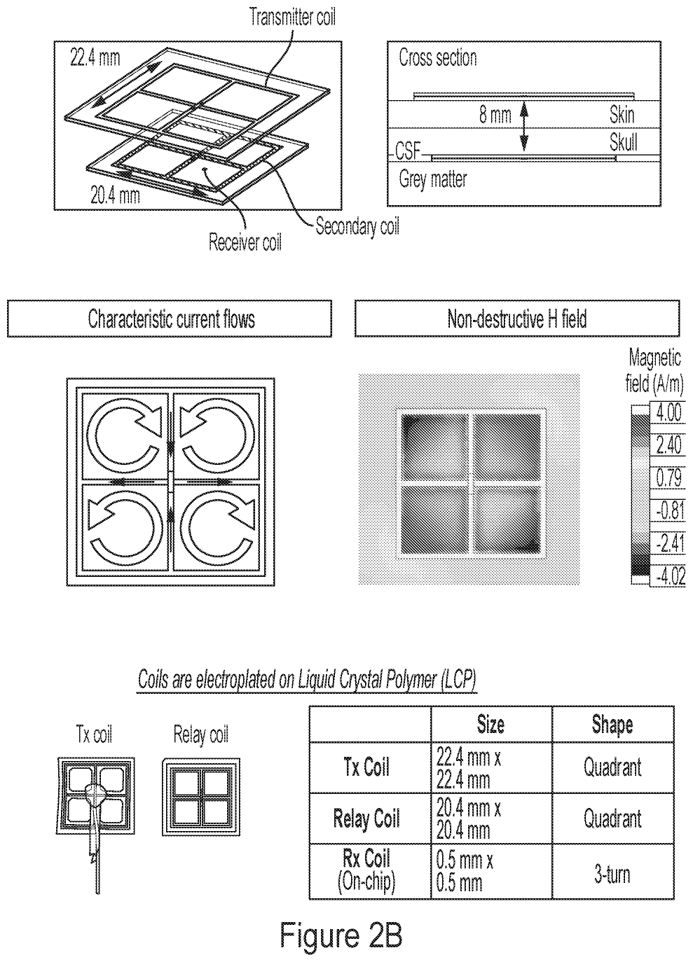

In FIG. 2A, an exemplary system flow is illustrated. Implanted NGs 20 wirelessly exchange information with a skinpatch 22 having a transceiver and demodulator. The skinpatch 22 provides neural signals to a wearable neurocoder 24 having neurocomputational algorithms for large scale cortical models. The wearable neurocoder 24 may provide encoded signals for patterned recognition to the skinpatch 22.

As shown in FIG. 2B, Tx and Relay design as area-matched 4-quadrant coils to maximize coverage area.

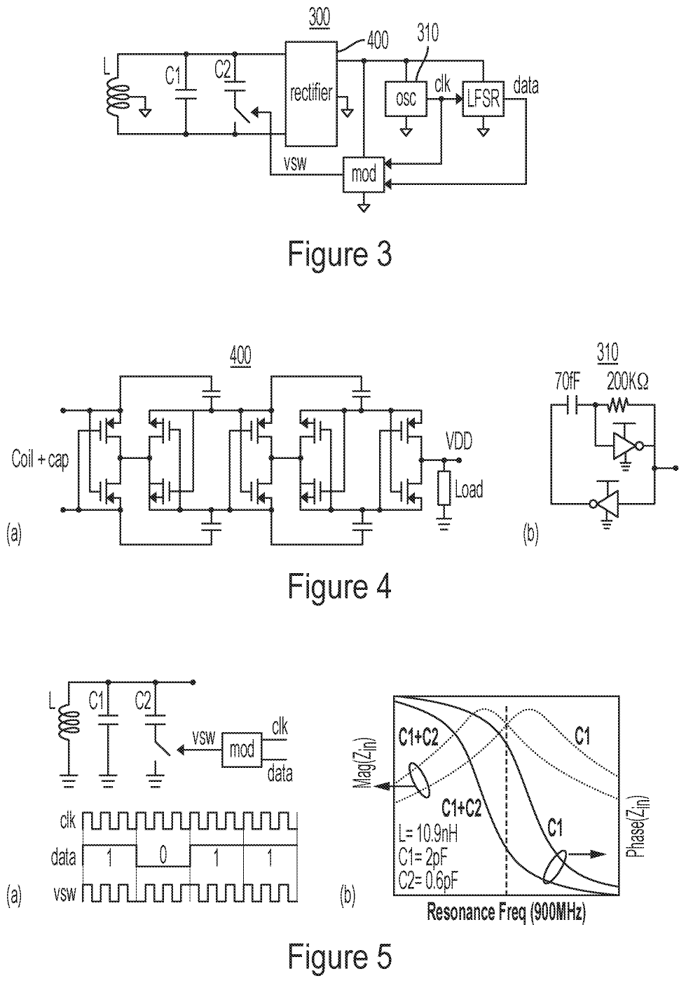

FIG. 3 shows a block diagram of an exemplary Neurograin ASIC 300. An on-chip coil (part of the 3-coil relay system) couples RF power to a rectifier 400, which then produces DC voltage to all on-chip circuits. The center-tap of the balanced coil serves as the on-chip ground reference (a “pseudo-ground” node). The oscillator 310 is designed to generate an ˜30 MHz clock signal upon free-running at start-up. For initial testing purposes, a 31-bit linear feedback shift register (LFSR) was used to produce a fixed uplink digital pattern at 10 Mbps. This data stream is converted into a Manchester-coded BPSK-encoded signal, which toggles a switchable capacitor in parallel to the on-chip inductor, thereby modulating the reflected RF waves (backscatter) in a data-dependent way to establish wireless uplink communication. Chip clock-frequency is chosen at 30 MHz to accommodate the 20 MHz BPSK backscattered spectrum for efficient RF demodulation/data recovery by the Skinpatch receiver.

This chip 300 provides an effective means to verify wireless power transfer and uplink communication performance. On-chip clock frequency can be accurately measured during data recovery, and given its known relationship to the rectifier output (on-chip VDD) through circuit simulations, we can measure wireless power transfer efficiency (PTE). Furthermore, the deterministic nature of uplink data (LFSR bit pattern) allows for link fidelity validation through computation of bit error rate (BER) from the measured received bits.

The rectifier circuit 400 is shown in FIG. 4(a). Maximizing the RF-to-dc power conversion efficiency has two challenges: (1) the rectifier needs to respond to small voltages received at the antenna (˜250 mVp), and (2) to achieve impedance matching so that the power available from the antenna is not reflected from the rectifier's high-impedance input.

First, we use low-VT NMOS transistors in a deep n-well available in the bulk 65 nm CMOS process to eliminate body effects. Second, the antenna-rectifier interface is co-optimized to achieve impedance matching. The coil is made multiple turns for higher (parallel) impedance (achieving L=10.9 nH, with Q=13). When resonated with C=2.3 pF, a bandwidth of 60 MHz is achieved. A multi (three)-stage rectifier design reduces its high input impedance by 9×. Simulations show that the RF-to-dc efficiency reaches a peak of 48.5% with an available RF power of −10 dBm at the Neurograin coil. The effective load impedance presented by the active circuitry is approximately 18.5 kOhm.

As shown in FIG. 4(b), an inverter-based relaxation oscillator 310 is chosen to generate the 30 MHz clock. The oscillation frequency is predominantly dictated by the RC time constant, with a secondary contribution from inverter transit time. The oscillator 310 may either be operated in a two-tone mode (injection-locked to the beat tone (f2−f1) generated by the rectifier) or the single-tone (free running) mode. Although the former approach provides a more stable (independent of process and coupled power variations) clock preferable for synchronized uplink/downlink communications, it also degrades the PTE by roughly 3 dB (in simulation) since the two RF tones are off-resonance. We have thus implemented the single-tone, higher-efficiency approach. A more sophisticated Skinpatch frequency/timing recovery function is designed to accommodate the variable Neurograin clock (28-33 MHz). A synchronized TDMA network can be achieved by an ASK-PWM downlink scheme.

The shared power/data telemetry inductive link between the neurograin and the skinpatch mandates that data communications occur without impacting wireless power transfer. This is achieved through BPSK modulation. Given the 10 Mbps uplink data, the modulator phase-shifts the 30 MHz clock by either 0° or 180° (FIG. 5a). C2 is designed such that when it is switched in and out of the on-chip resonant network (L, C1), the magnitude of the input impedance remains constant (thus maintaining power transfer), while the phase difference is maximized (for effective backscattering). This is illustrated in FIG. 5(b). Overall phase variation between the two states is approximately 140 degrees for nominal frequencies (and decreases for frequency mistuning).

Two neurograin test chips have been designed and fabricated in TSMC 65 nm CMOS LP process. One features continuous (non-stop) backscatter of LFSR data (effective for power efficiency and single-channel backscatter evaluations), while a second includes additional digital circuits to perform “packetized” backscatter (100 μs packets at 100 ms periodicity). Physically unclonable functions (PUFs) are integrated onto the latter to randomize the backscatter start time per chip. These chips are spatially distributed in dense clusters and wirelessly interfaced with a single skinpatch antenna to form a multi-channel neurograin network.

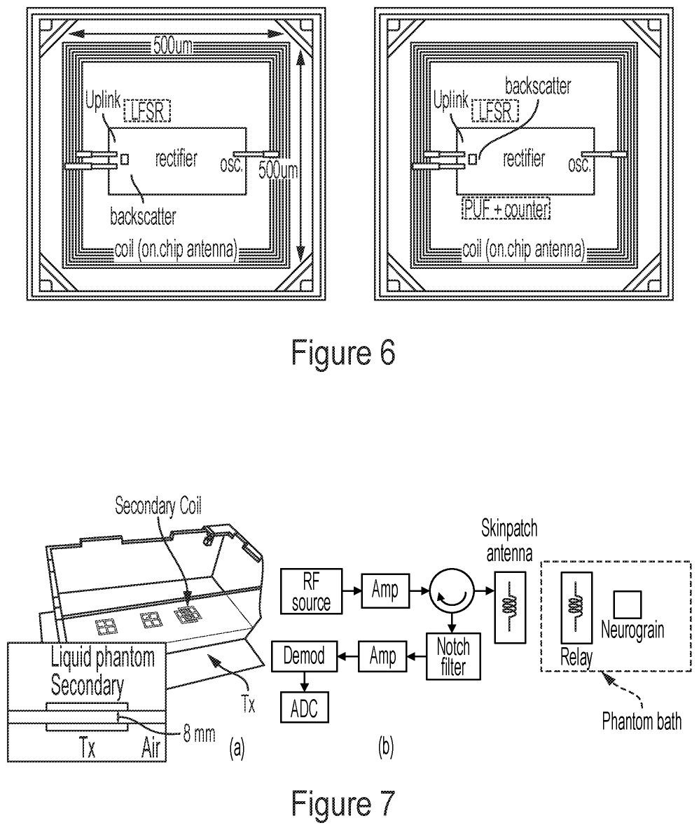

FIG. 6 illustrates photomicrographs of the test chips. The outer coil dimension is 500 by 500 μm2, while the active circuits measure ˜300 by 150 μm2. The on-chip antenna is realized with ultra-thick (3.4 μm) top copper. Layout optimizations for improved energy harvesting include placement of active circuits with ample spacing from coil, and breaking/slotting metal planes and routings to avoid formation of eddy current loops which may de-Q the on-chip antenna.

To model realistic tissue loss and the surrounding permittivity, the continuous-backscatter chip is hermetically sealed in liquid crystal polymer (LCP), and tested in a liquid brain phantom, with 8 mm gap between Tx and secondary coils, as shown in FIG. 7(a).

The skinpatch antenna is driven with ˜30 dBm of 915 MHz RF power through a microwave circulator, which also isolates the Neurograin backscattered signal (FIG. 7(b)). This received signal is notch-filtered (to reject the leaked Tx tone) and down-converted to baseband.

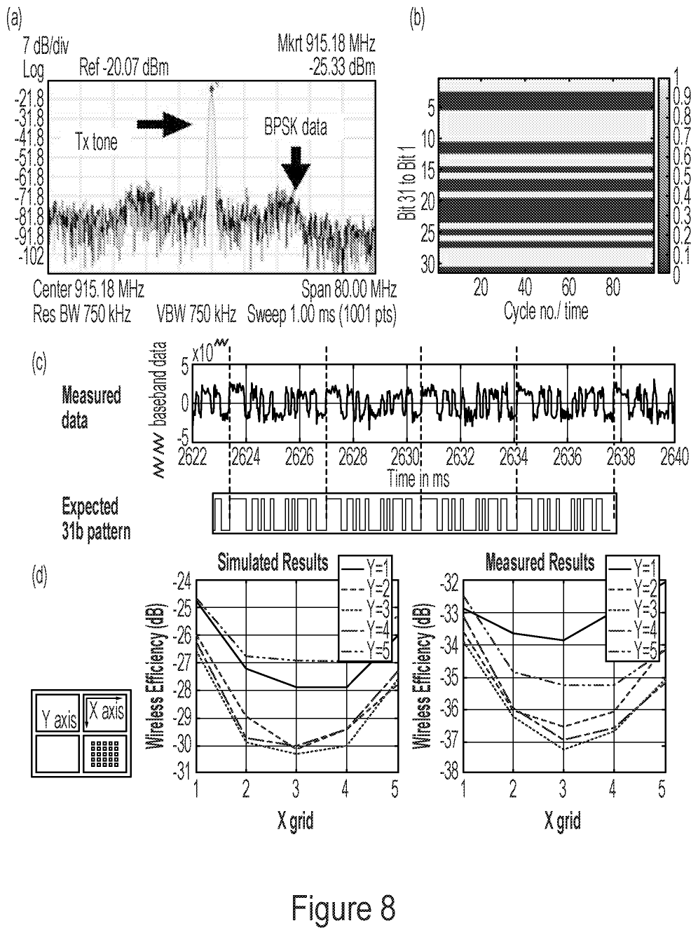

The measured RF spectrum from a single backscattering neurograin is illustrated in FIG. 8(a). Center frequency is 915 MHz, with backscattered BSPK spectrum clearly visible. The frequency separation between the BPSK spectrum and the Tx tone indicates the on-chip clock frequency, which in turn is utilized to evaluate the PTE. The spectrum is downconverted and demodulated to recover the received bit-stream, which is cross-validated against the pre-designed 31b LFSR pattern in FIG. 8(c). FIG. 8(b) illustrates a long received bit-stream in a color coded 2-D map, showing no errors. We are able to consistently achieve 0% BER at the strongest-coupling locations (along the relay perimeter), and maintain <1% BER at all locations.

From circuit simulations, we can find the relationship between the available power (from −10 to −5 dBm) at the on-chip coil versus the oscillator frequency (from 23 to 34 MHz). Therefore, by measuring the clock of a neurograin at 25 locations, we can find the spatial PTE. While the trend of measured PTE versus position matches our EM simulations, roughly 7 dB higher source power is required in the current experimental setup (FIG. 8(d)). This is attributed to higher electrical conductivity of the liquid phantom and/or impedance mismatch on Tx and relay coils.

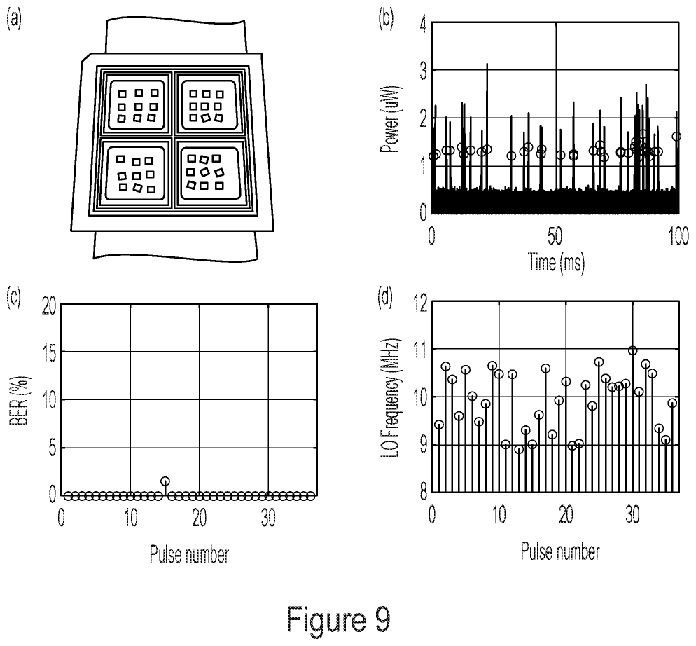

This version of neurograin chip backscatters 100 μs packets once every 100 ms (frame) period, with randomized start times to enable the formation of a multi-channel random access uplink communication network. Experimentally, 36 chips were placed in a distributed manner within a relay coil, as shown in FIG. 9(a). Measurement was conducted in air. In 100 ms, 36 isolated packets were recovered, thus confirming the network's functionality. FIGS. 9(b)-9(d) summarizes the measured received power, BER and clock rate per received packet. Extension of this network is limited only by the increase in BER due to possibility of packet collisions. For this asynchronous communications protocol, ˜100 chips may be networked with an acceptable packet collision rate.

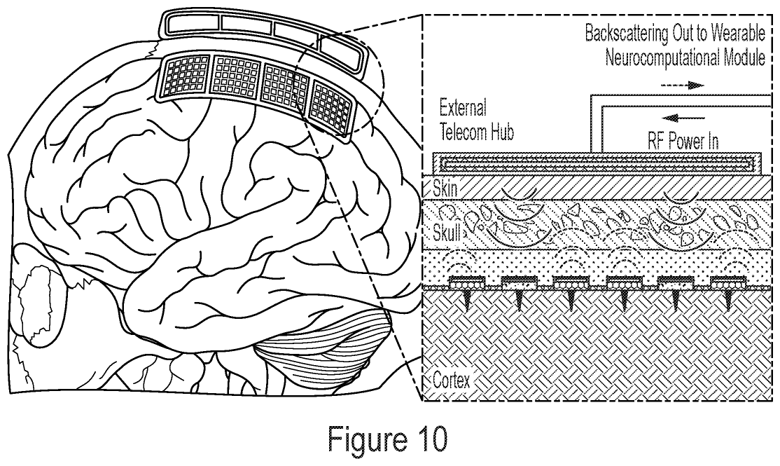

In summary, FIG. 10 illustrates an overview of a spacially-distributed cluster of adaptive, implanted neurograins and external telecommunications hub leading to a wearable neurocomputational unit (not shown).

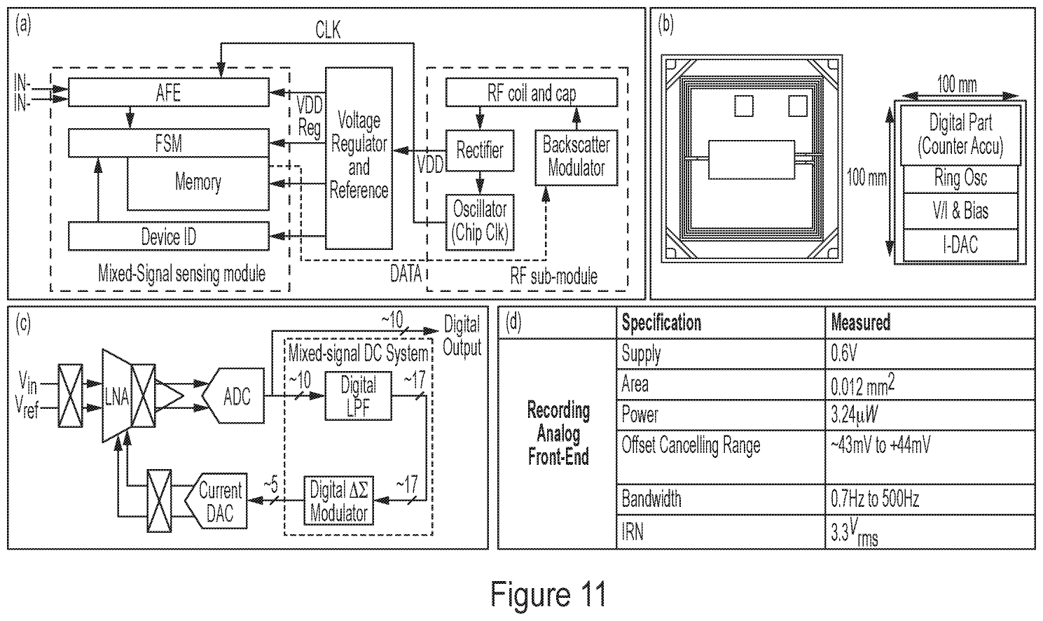

FIG. 11(a) illustrates a top level schematic of a wireless recording neurograin, featuring an AFE configured for epicortical recording.

FIG. 11(b) illustrates an exemplary photomicrograph and layout of Recording ASIC.

FIG. 11(c) illustrates an exemplary circuit block diagram for recording Neurograin Test ASIC AFE, which FIG. 11(d) provides a summary of key performance metrics from bench top testing of our design.

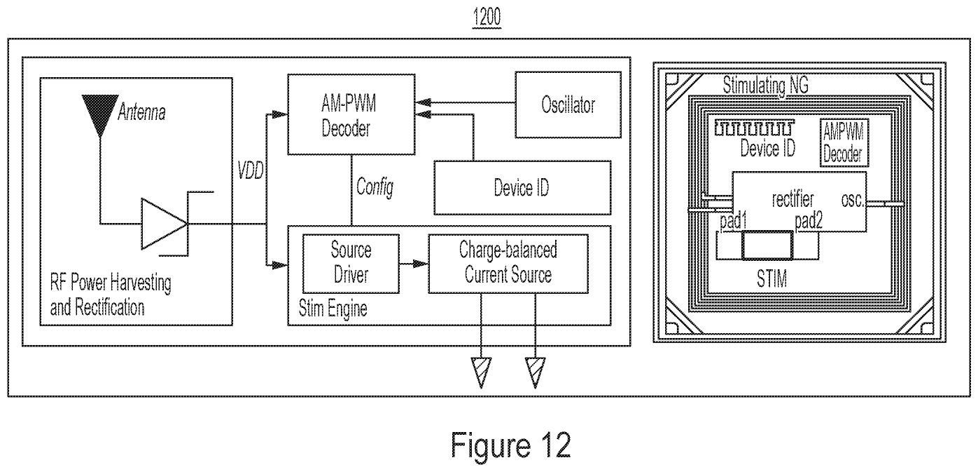

FIG. 12 illustrates a top level block diagram 1200 of a wireless stimulating neurograin, featuring a full RF energy harvesting module, a telemetry link and a programmable current source (Left) and a photomicrograph (Right).

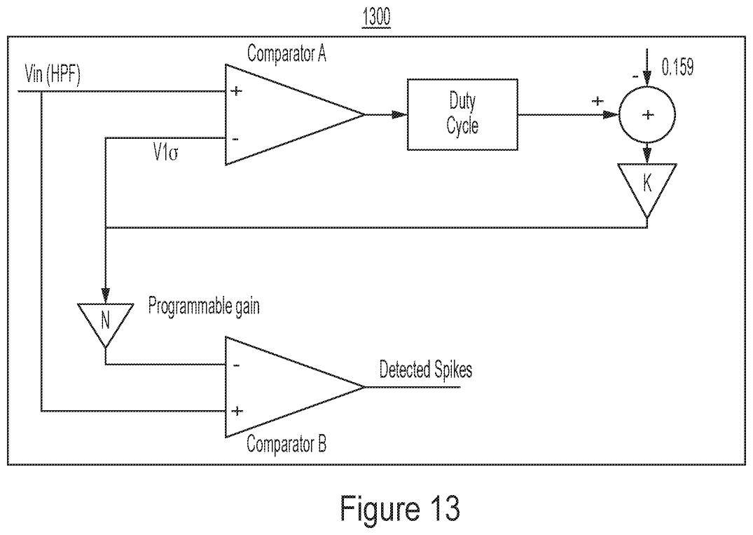

FIG. 13 illustrates an overview block diagram 1300 for an implementation of analog Gaussian-noise variance based voltage thresholding spike detection circuit.

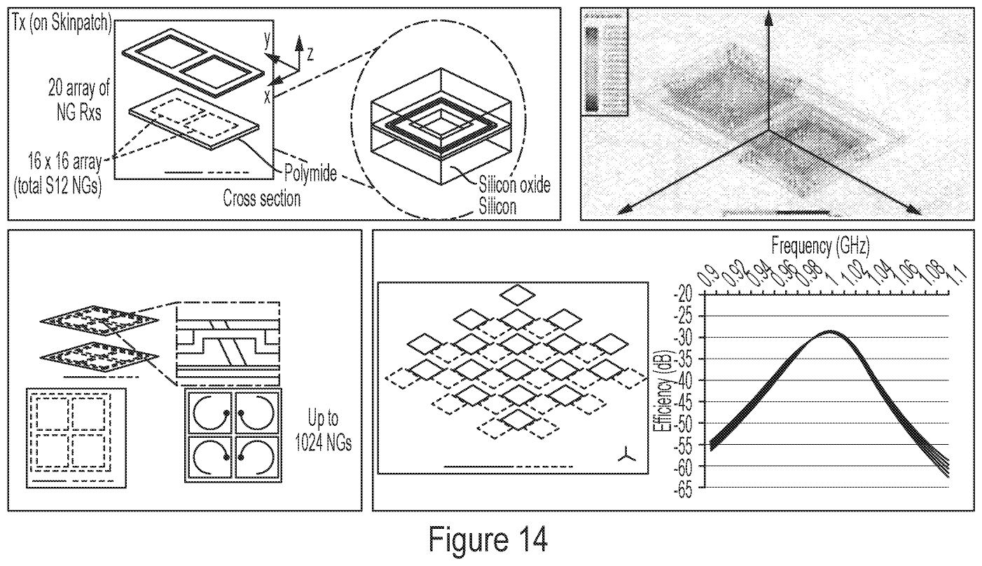

FIG. 14 illustrates an exemplary wireless power transfer for a neurograin system. Top left shows the WPT system architecture with a thin-layer Cu-based transmit, and mating relay coil antennas. These are based on flexible PCB substrate in current designs. Individual Neurograin can be successfully powered anywhere within the relay coil cross section area Top Right shows results from electromagnetic simulations, demonstrating magnetic flux across coil cross-section. Bottom left demonstrates the most-current coil topology with window-shaped relay designed to mitigate destructive field effects, and Bottom right shows optimized performance at 1 GHz of a staggered array of tightly packed neurograins coils.

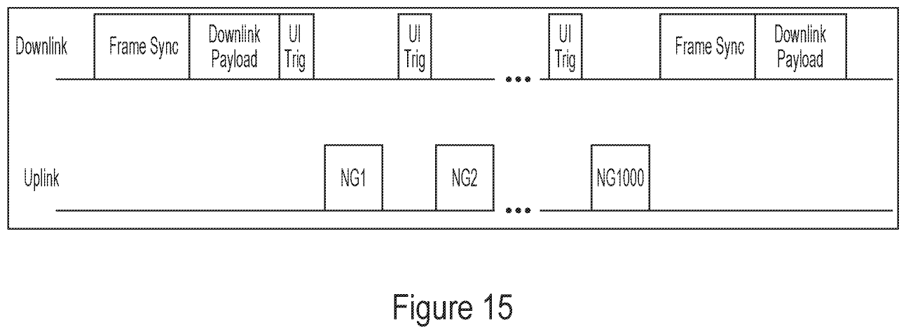

FIG. 15 illustrates a time-division multiple access RF communication scheme. Each frame starts with a frame sync, followed by a downlink payload, which custom configures each network node with respect to its status. Each node is pre-assigned a slot in the transmission queue using its unique hardware ID.

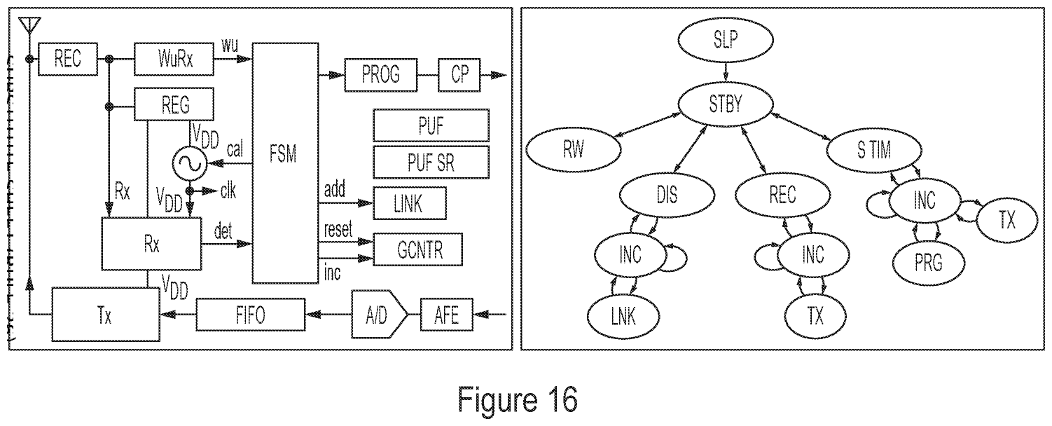

In FIG. 16, an overview of a fSM implemented on a neurograin ASIC is shown.

In other implementations, we have developed techniques using biodissolvable delivery devices for high-throughput intracortical delivery.

In still other implementations, the neurograins can be applied to “read-out” (=record) and “write-in” (=stimulate) neurologically and physiologically relevant spatio-temporally patterned electrical, optical, magnetic, and ultrasound signals, whether in the brain or the peripheral nervous system.

In summary, untethered microelectronic chiplets (referred to herein as “neurograins”) form a huge network of individual neural interfacing nodes in the cortex for active neural recording and electrical microstimulation. An individual neurograin on the scale of 100 microns in size integrates a radio frequency (RF) power-harvesting circuit, neural sensing microelectronics and sophisticated telecommunications solutions at the cutting-edge CMOS technology, and is hermetically sealed for long-term reliability when implanted. External electronics on a skinpatch enable wireless powering, real-time read-out of neural data, and write-in of neural modulation on a timescale of less than 1 ms from a thousand spatially distributed Neurograins.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A system comprising:
a distributed sensor system of complementary metal-oxide-semiconductor (CMOS) untethered microelectronic chiplets forming a network of individual neural interfacing nodes in a cortex for active neural recording and electrical microstimulation.

2. The system of claim 1 wherein each of the microelectronic chiplets comprises a hermetically sealed unit including a radio frequency (RF) power-harvesting circuit, neural sensing microelectronics and a telecommunications circuit.

3. The system of claim 2 further comprising a skin patch, the skin patch including external electronics to enable wireless powering of the microelectronic chiplets, real-time read-out of neural data, and write-in of neural modulation.

4. A system comprising:
a distributed sensor system of CMOS neurograins that provide a high density network of autonomous implantable neural sensors.

5. The system of claim 4 wherein the CMOS neurograins are organized into a two-dimensional (2-D) grid that freely float in a cerebrospinal fluid (CSF) surrounding a human brain.

6. The system of claim 4 wherein each of the CMOS neurograins comprises a dedicated radio frequency (RF) micro-antenna, an analog front-end circuit, a networking circuit and backscatter circuit.

7. The system of claim 6 wherein each of the CMOS neurograins is powered by inductive coupling with an antenna contained in an epidermal skinpatch.

8. A system comprising:
an implanted autonomous microchiplet communicatively linked to an epidermal skinpatch having a transceiver and a demodulator;
an intermediate device communicatively linked to the epidermal skinpatch, the intermediate device configured to receive neural signals for decoding from the epidermal skinpatch and to send encoded signals for patterned stimulation to the epidermal skinpatch; and
one or more external devices communicatively linked to intermediate device.

9. The system of claim 8 wherein the external device is a cloud computing node.

10. The system of claim 8 wherein the external device is a smart effector device.

11. The system of claim 8 wherein the external device is a prosthesis.

12. The system of claim 2 wherein the unit is hermetically sealed unit with on or more thin-layer dielectric coatings.
